(19) United States
(12) Patent Application Publication
(10) Pub. No.: US 2024/0139452 A1
(43) Pub. Date: May 2, 2024

(54) APPARATUSES AND METHODS FOR INTUBATION

(71) Applicant: Airway Management Technology LLC, Palo Alto, CA (US)

(72) Inventors: Babak JAMSHIDI, Palo Alto, CA (US); Alexander HOGHOOGHI, Palo Alto, CA (US)

(21) Appl. No.: 18/499,056

(22) Filed: Oct. 31, 2023

Related U.S. Application Data
(60) Provisional application No. 63/421,063, filed on Oct. 31, 2022.

(51) Int. Cl. A61M 16/04; A61B 1/00; A61B 1/005; A61B 1/267

(52) U.S. Cl. CPC A61M 16/0488; A61B 1/00045; A61B 1/0052; A61B 1/267

(57) ABSTRACT

Described herein are apparatuses for guiding intubation procedures, and methods of intubating a patient using them. The methods and apparatuses described herein provide systems that may be rapidly and safely deployed to safely and rapidly target and deliver intubation. For example, any of these systems may include an endotube introducer having an elongate body and a bend region, a camera at a distal end region, and a bend control that is configured to alter the bend of the bend region. The endotube introducer may be pre-loaded with an intubation tube within a channel or lumen of the insertion endoscope. Also described herein are methods of using these apparatuses to rapidly and safely position and secure an endotracheal tube within a patient's trachea.

### CLAIM OF PRIORITY

This patent application claims priority to U.S. Provisional Patent Application No. 63/421,063, titled “APPARATUSES AND METHODS FOR INTUBATION,” filed on Oct. 31, 2022, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

The methods and apparatuses described herein may be related to trans-oral endoscope systems for guiding intubation procedures and methods of intubating a patient using them. More specifically, the methods and apparatuses described herein may relate to apparatuses that may use a system that may be rapidly and safely deployed to form a stable platform for targeting and delivery of intubation.

### BACKGROUND

Endotracheal intubation is a common emergency procedure to provide an alternative airway to a patient whose airway may be compromised or blocked. This procedure may be performed in the operating room setting, outside of the operating room, in the course of care within the emergency department, and even out in the field. Despite the fact that these procedures are performed with some frequency, endotracheal intubation remains a high-risk procedure, with a significant rate of respiratory complications, hemodynamic instability, and cardiac arrest. Any new technology that improves the safety and success of endotracheal intubation can therefore have a tremendous effect on public health and safety.

Described herein are methods and apparatuses (e.g., systems) for performing endotracheal intubation that may improve the speed and safety of these procedures.

### SUMMARY OF THE DISCLOSURE

The methods and apparatuses (e.g., systems and devices, including software, hardware and firmware) of this disclosure each have several innovate aspects, no single of which is solely responsible for the desirable attributes disclosed herein. In general, these apparatuses may be used to perform endotracheal intubation in a manner that is noticeably faster, e.g., in most cases, in under 60 seconds, and safer, preventing damage to the patient's mouth and teeth.

One innovative aspect of the subject matter described in this disclosure can be implemented as an apparatus for intubating a patient that may include an endotracheal introducer. In general the endotube introducer may be preloaded with an endotracheal tube that can be deployed distally out of the endotube introducer once it is in the correct position. The endotube introducer may include an elongate body that has a bent or curved region midway down the length of the elongate body; the bend/curvature of this region may be controllably increased either manually, automatically or semi-automatically to position the distal end of the endotube introducer opposite the epiglottis so that the pre-loaded endotracheal tube may be extended distally into the trachea, where it can be anchored in position. The individual components or features of any of these systems may themselves be patentable and may include a number of useful improvements and advances in the art.

For example, described herein are apparatuses (e.g., systems and devices) comprising: an endotube introducer configured to be inserted through an oral cavity into a tracheal region of a body, the endotube introducer comprising an elongate body having a lumen extending therethrough and a bend region on the elongate body, a camera at a distal end region, and a bend control at a proximal end region configured to alter the bend of the bend region; a display configured to receive and display output from the camera; an intubation tube preloaded within the lumen of the insertion endoscope, the intubation tube having an expandable anchor at a proximal end region, wherein the intubation tube is configured to be advanced distally out of the endotube introducer.

In some examples the apparatuses (e.g., systems) described herein may include: an endotube introducer configured to be inserted through an oral cavity into a tracheal region of a body, the endotube introducer comprising an elongate body having a lumen extending therethrough and a bend region between 7 cm and 20 cm from a distal end of the elongate body that is configured to increase a bend of the elongate body to 70 degrees or more relative to the longitudinal axis of the elongate body, a camera at a distal end region, and a bend control at a proximal end region configured to bend or straighten the elongate body at the bend region; and a display configured to receive and display output from the camera; wherein the lumen is configured to slidably hold an intubation tube preloaded within the lumen of the insertion endoscope so that the intubation tube may be advanced distally out of the endotube introducer.

The elongate body of the endotube introducer may be prebend or curved, typically at a region that is approximately midway down the length of the elongate body of the endotube introducer. The position of the bend may be based on an optimal position (based on population data) so that the distal end region may be inserted to the epiglottis and may guide the distal end region of the endotube introducer into the trachea, so that the endotracheal tube may be extended distally into the trachea for rapid anchoring. Although in some cases the dimensions of the endotube introducer may be configured to a variety of different patients, in some cases the endotube introducers described herein may be configured to be sized for small (e.g., infants/children), medium (smaller patents) and/or large (e.g., adult) patients. In some examples, the bend region of the endotube introducer may be between about 5 cm and about 25 cm (e.g., between about 7 cm and about 20 cm, about 8 cm and about 18 cm, etc.) from the distal end of the endotracheal introducer. In any of these apparatuses the elongate body of the endotube introducer may have an initial, at rest, configuration in which the bend region is pre-bend between about 25 and about 50 degrees. Thus, the bend region may have a minimum set bend of between about 25 and 50 degrees.

In general, the bend control may any appropriate control, including but not limited to a dial, knob, slider, etc. The bend control may be configured to increase or decrease the bend of the bend region; for example, the bend control may increase the bend of the bend region up to about 70 degrees or more relative to a longitudinal axis of the elongate body. The bend region may be actuated by the bend control by pulling a tendon or tendons (e.g., one or more wires, cables, etc.) to drive bending of the bend region.

In any of the endotube introducers described herein, the distal end of the endotube introducer may be configured to be driven against the laryngeal surface of the epiglottis with sufficient force to elevate the epiglottis and open the airway to allow passage of the endotracheal tube into the trachea. Thus, any of these apparatuses may be configured so that the endotube introducer, and in particular the bend region (e.g., actuated by the bend controller) is configured to bend the bend region against two (2) or more Newtons (N) of force (e.g., 3 N or more, 4 N or more, 5 N or more, 6 N or more, 7 N or more, 8 N or more, 9 N or more, 10 N or more, etc.).

In general, the distal end region of the endotube introducer may be adapted both to insert into the body and/or to apply force against the laryngeal surface of the epiglottis. For example, the distal end region of the endotube introducer may include one or more projections configured to atraumatically displace tissue. In some cases the distal end region may include a tapered region that is formed of a relatively soft (e.g., having a lower durometer than more proximal regions) material. The distal end region may have a durometer of less than 70 (e.g., less than 60, less than 50, less than 40, less than 30, less than 20, etc.) on the Shore A scale. The distal end region may have curved and/or rounded edges. The distal end region may extend as a rim or ridge. In any of these apparatuses the bend region may bend in just one plane, e.g., the plane of the elongate body of the endotube introducer. Thus, the end region may be constrained to bend in a first plane that is parallel with the elongate body.

Any of these apparatuses may be configured to output, in real time, images from the distal end region of the endotube introducer, which may be useful to navigate and/or deploy the endotube introducer and/or the endotracheal tube. In any of these apparatuses the endotube introducer may include a camera, e.g., facing distally of the distal end region. In some example the endotube introducer may include one or more cameras that are configured to display visual information of the distal end region as the endotube introducer is advanced into the body. The apparatus may be configured to continuously display video images looking distally from the distal end region of the apparatus. The endotube introducer may be wired or wirelessly connect the camera of the endotube introducer to one or more displays. The display may be associated with the endotube introducer, or with a device in communication with the endotube introducer. In some examples the endotube introducer may be in communication with a communications device (e.g., smartphone, tablet, laptop, etc.). In some examples, the apparatus is configured to be in communication with a dedicated display.

Any of these apparatuses may be configured so that the endotube introducer may be removed from the body and separated from the endotracheal tube once the endotracheal tube is positioned and anchored within the trachea. In some examples the endotube introducer may be removed over the distal end of the endotracheal tube by withdrawing the endotube introducer proximally after anchoring the endotracheal tube. In any of these apparatuses the endotube introducer may be removed through a channel, slit, longitudinal side opening, etc. through the distal end region of the endotube introducer. Since, in some examples, the more proximal end of the endotracheal tube may have a narrower diameter than the more distal region(s) of the endotracheal tube, withdrawing the endotube introducer at least partially proximally may allow the endotracheal tube (and in some examples the inflation tube coupled to the anchor, e.g., balloon) to exist a longitudinal side opening through the body and into the lumen.

Any of the apparatuses described herein may be configured to include one or more processors that include software, hardware and/or firmware to assist in navigation and/or control of the endotube introducer for positioning and deploying the endotracheal tube. The apparatuses described herein may include a software agent that is trained to recommend and/or control operation of the endotube introducer, including steering the endotube introducer. For example, any of these apparatuses may include a trained machine learning agent that is trained to recognize (e.g., from the one or more camera inputs) the laryngeal surface of the epiglottis, confirm orientation of the distal end region of the endotube introducer, and either (or both) indicate that the bend region should be actuated to apply force from the distal end region to elevate the epiglottis and opening the airway, and/or may automatically (or semi-automatically, after confirmation from the user) increase the bend angle of the bend region and apply the force to elevate the epiglottis and open the airway.

For example, any of these apparatuses may include one or more processors, wherein the one or ore more processors comprises a non-transitory computer-readable medium including instructions that, when executed by the one or more processors, cause the one or more processors to perform a method comprising: presenting guidance information on the display to guide the user to insert the endotube introducer. In some examples the or ore more processors comprises a non-transitory computer-readable medium including instructions that, when executed by the one or more processors, cause the one or more processors to perform a method comprising: activating a control on the steerable endotube introducer to increase a bend of the elongate body so that the distal end region elevates the epiglottis. Thereafter the endotracheal tube may be advanced distally into the trachea and anchored.

Also described herein are methods of using any of these apparatuses. For example, a method of inserting an intubation tube may include: inserting a distal end region of an endotube introducer having an elongate body through to a patient's mouth, wherein the endotube introducer is preloaded with an endotracheal tube; advancing the distal end region of the endotube introducer into the patient, while imaging a region distal to the distal end region from a camera on the distal end region, to position the distal end region beneath a laryngeal surface of an epiglottis; activating a control on the steerable endotube introducer to increase a bend of the elongate body so that the distal end region elevates the epiglottis; advancing the endotracheal tube distally out of the endotube introducer and into the patient's trachea; anchoring the endotracheal tube within the trachea; and removing the steerable endotube introducer from the patient leaving the endotracheal tube within the trachea.

In some examples the method may include: inserting a distal end region of an endotube introducer having an elongate body through to a patient's mouth, wherein the endotube introducer is preloaded with an endotracheal tube; advancing the distal end region of the endotube introducer into the patient, while imaging a region distal to the distal end region from a camera on the distal end region, to position the distal end region beneath a laryngeal surface of an epiglottis, wherein a control on the steerable endotube introducer is controlled to increase a bend of the elongate body so that the distal end region elevates the epiglottis; advancing the endotracheal tube distally out of the endotube introducer and into the patient's trachea; anchoring the endotracheal tube within the trachea; and removing the steerable endotube introducer from the patient leaving the endotracheal tube within the trachea.

In general anchoring may include expanding an expandable balloon. Alternatively anchoring may include expanding a mechanical anchor (e.g., basket, stent, etc.). The endotracheal tube may generally be connected, either before, during or after insertion, to a source of air (e.g., an air circuit, pump, respirator, ventilator, pump, etc.). For example, any of these methods may include connecting the endotracheal tube to an air circuit.

In any of these methods, the method may include activating the control to increase the bend of the elongate body from an angle of between 25 and 50 degrees relative to a longitudinal axis of the elongate body to an angle of greater than 70 degrees relative to the longitudinal axis of the elongate body. Similarly, activating the control may comprise increasing the bend of the elongate body at a bend region that is between 7 cm and 20 cm from a distal end of the elongate body. Any of these method may include one or more controls for controlling assisting, using a guidance sub-system associated with the endotube introducer, in controlling the positioning of the distal end region.

Any of the apparatuses (e.g., system, devices, etc.) described herein for intubating a patient may include an oral retractor (“mouth opener” or “jaw opener”) that may secure a patient's jaws in an open position to allow safe insertion of one or more medical devices, including an intubation tube. The oral retractor may be configured for easy and rapid deployment into the patient's oral cavity to hold the mouth open. In particular, the oral retractor may be configured to hold the mouth open so that the teeth and/or tongue are protected and prevented from damage by insertion of one or more medical devices through the opening formed by the oral retractor. In some examples a portion of the endotube introducer (e.g., a base region) may be configured as an oral retractor.

For example, any of these apparatuses may also include a base (e.g., “entry base” or stand, or base region) that may securely couple to the oral retractor. In some examples the base may be integral and/or fixed to the oral retractor. In some examples the base may be removably coupled to the oral retractor. For example, the base may be configured to couple to the oral retractor after the oral retractor is inserted into a patient's oral cavity and may snap or lock into position over the oral retractor. The base may include a support or platform portion to which additional structures (as described herein) may be securely coupled. The base may include one or more openings (a central oral opening, referred to herein as a tube guide entry) into the patient's mouth that may allow passage of one or more medical devices, including, as will be described in greater detail below, an endotube introducer and an intubation tube. In some cases the tube guide entry may be separate or separable and may be coupled to the base. In some examples the tube guide entry may be formed as or included as part of the base.

The apparatuses described herein may also include one or more displays (e.g., liquid crystal displays, or LCDs, or other displays). In some examples the display may be configured to removable couple to the base to allow the clinician (doctor, nurse, technician, etc.) to view the display while operating the system to intubate a patient. The display may connect wireless or via one or more cables or wires to a medical device (e.g., endotube introducer, bougie tube, etc.) to display one or more images taken by the medical device. This may assist the clinician in operating the system. In some example the display may also act as (or may be coupled with) an input such as a touchscreen display. The display may also include one or more controls (inputs) for operating the display and/or other portions of the system. The display may be coupled to the base either mechanically or electrically (e.g., magnetically) or both.

As mentioned any of these apparatuses may generally include an endotube introducer. The endotube introducer may include a bent and steerable (at least at the distal region) elongate body that may be inserted into the patient's oral cavity and be positioned within the oral cavity and steered to or near the trachea. The endotube introducer may be configured as a flexible endoscope with one or more (preferably two) cameras for imaging from the distal end region of the endotube introducer, one or more light sources (e.g., light emitting diodes, LEDs), and one or more built-in suction tubes. The endotube introducer may include one or more controls for steering the distal end region of the device. For example, the endotube introducer may include one or more tendons that actuate the bending of the distal end of the device from a proximal control. The bend control (also referred to herein as a proximal control) may be a knob or dial, or more than one knob or dial that control tension on the tendons articulating the distal end, to steer the device.

In general, an endotube introducer apparatus may be configured as a hollow tubular body. The distal end of the endotube introducer may preferably be covered by two or more flaps or facets that are normally closed but may be controllably opened when the endotube introducer is positioned at or near the trachea. The flaps or facets may be opened outwards in order to displace the surrounding tissue without damaging it, in order to make room for the intubation tube and make it easier to visualize and insert the intubation tube. The endotube introducer body may have a plurality of peripheral channels for holding one or more cameras, suction/vacuum lines, light source(s), and/or drivers for driving opening of the flaps at the distal end of the endotube introducer apparatus.

In some examples, the endotube introducer (herein equivalently referred to as simply an endotube) may be configured to interact with the base so as to allow the endotube introducer to be inserted and manipulated (rotated, tilted, advanced/retracted, etc.) freely through the base, but may be locked and secured in position once the endotube introducer has been located at or near the trachea in the proper orientation. For example, any of these components, including the endotube introducer, may be securely and quickly coupled to (or locked to) the base, as by magnets, mechanical couplers, etc.

Any of these apparatuses may also include one or more endotracheal tubes (e.g., intubation tubes). Standard or customized intubation tubes may be used. In particular, the intubation tubes described herein may have a length that is greater than 24 inches (e.g., 30 inches, 32 inches, 34 inches, 36 inches, etc.). The longer (nonstandard) lengths described herein may be particularly well adapted for use with the systems described herein. The intubation tube may include a passage for air and/or a passage for activating an anchor (e.g., anchoring balloon) to secure the intubation tube in place. In some examples the anchor may be an inflatable balloon.

Any of these apparatuses may also include a tool for removal of the endotube introducer or other components, while leaving the intubation tube in place and undisturbed. In some examples the endotube introducer may be removed using a removal tool referred to herein as an endoscopic bougie tube, or simply a bougie. The bougie may be configured to be applied within and/or over the intubation tube and within the endotube introducer to allow the endotube introducer (and base, and oral retractor) to be removed without disturbing the intubation tube.

Also described herein are methods of operating the system in order to insert an intubation tube. In general, the method may be performed while the patent lays on his or her back (and may include laying the patient on their back). The patient's jaws may be held open by the oral retractor (jaw retractor). The base may be either pre-coupled (or integrally attached) to the oral retractor, or it may be coupled after attaching the oral retractor to the patient. The display may then be coupled to the base to allow visualization from the display.

In some example, in which a separate tube guide entry is used the tube guide entry may be coupled to the base. The endotube introducer may be coupled to the display (wireless or by one or more wires or cords) and may be inserted into the oral cavity through the tube guide entry. The endotube introducer may be inserted until the distal end (which in some examples may be closed by the flaps or facets) is proximal to the trachea. For example, the endotube introducer may be inserted and steered by the proximal control, using visualization from the display, until it reaches the epiglottis; the tip may then be adjusted until it faces the trachea. In some examples the flaps or facets may be opened to displace tissue without damaging it and making the trachea easier to visualize and/or access.

The intubation tube may then be inserted through the endotube introducer until it passes the tip of the camera(s) on the endotube introducer and is inserted into the trachea. The inflatable or expandable anchor may then be engaged. Once the patient's vitals are stable, the system components may be removed from around the intubation tube. In some examples, a tool (e.g., bougie) may be inserted, optionally after coupling to the display, into the endotube introducer and over and/or within at least a portion of the intubation tube to support the intubation tube and allow the endotube introducer, base and jaw retractor from the patient, leaving the intubation tube behind. The patient's vitals may be continuously monitored before, during and after this process. In some examples the endotube introducer may be removed without the use of a separate tool. For example, the endotube introducer may be configured to be withdrawn proximally over the endotracheal tube (e.g., intubation tube) once it is anchored in place in the trachea, and the proximal region of the endotracheal tube can be removed via a channel, slot or other opening along the length of the endotube introducer.

In some examples described herein the apparatus (e.g., system) includes: an oral retractor; a base coupled or couplable to the oral retractor including an opening through the base into an oral cavity; a display coupled securely to the base; an endotube introducer configured to be inserted through the opening through the base and into the oral cavity, wherein the endotube introducer comprises one or more cameras in communication with the display, wherein the endotube introducer and has a steerable distal end; an intubation tube configured to be inserted through the endotube introducer; and a bougie configured to be inserted through the endotube introducer to stabilize the intubation tube while the endotube introducer, display, base and oral retractor are removed.

The base may be integral to the oral retractor. In some examples, the base is locked onto the oral retractor. Alternatively, the base coupled to one or more connectors on the oral retractor.

The endotube introducer may include a plurality of facets over the steerable distal end opening that are controllable opened to displace tissue. The facets may be leaflets, flaps, covers, etc., and may be non-traumatic (e.g., not sharp and/or rounded, etc.) and may cover the distal end opening into the endotube introducer to prevent damage to the lumen when inserting and steering the apparatus. The facets may be flexible or stiff and may be configured to push tissue away from the distal end opening so that structures, including but not limited to the trachea, may be more easily visualized and/or accessed.

The bougie may comprises an elongate, tubular body, and in some examples, may include a camera configured to wirelessly couple the bougie to the display.

The endotube introducer may be wirelessly coupled to the display, e.g., to display images and/or video from the endotube introducer on the display.

In general, the oral retractor may be configured to include a first mouthpiece and a second mouthpiece that are each configured to enclose at least a buccal side of a patient's teeth. In any of these oral retractor described herein, one or both of the first mouthpiece and the second mouthpiece may be adjustably coupled to the oral retractor.

Any of these apparatuses may include a lock, e.g., a lock configured to lock a position of the endotube introducer relative to the base.

For example, a system may include: an oral retractor; a base coupled or couplable to the oral retractor including an opening through the base into an oral cavity; a display coupled securely to the base; an endotube introducer configured to be inserted through the opening through the base and into the oral cavity, wherein the endotube introducer comprises one or more cameras in communication with the display, wherein the endotube introducer and has a steerable distal end, wherein the steerable distal end is covered by one or more facets; a control coupled to the steerable distal end of the endotube introducer, and second control coupled to the one or more facets and configured to actuate the one or more facets; an intubation tube configured to be inserted through the endotube introducer; and a bougie configured to be inserted through the endotube introducer to stabilize the intubation tube while the endotube introducer, display, base and oral retractor are removed.

Also described herein are methods. For example, described herein are methods of inserting an intubation tube, the method comprising: attaching an oral retractor and base to a patient's mouth to retract mouth open and establish a stabilizing base; inserting a steerable endotube introducer through an opening in the stabilizing base and steer a distal end of the steerable endotube introducer adjacent to the patient's trachea while displaying images from a camera on the steerable endotube introducer on a display mounted to the stabilizing base; deploying the steerable endotube introducer to extend facets from a distal end of the steerable endotube introducer; and inserting an intubation tube through the steerable endotube introducer and into the patient's trachea.

Inserting the steerable endotube introducer may include protecting the patient's teeth with one or more mouthpieces on the oral retractor. Attaching the oral retractor and the base may include coupling the base to the oral retractor after the oral retractor is inserted into the patient's mouth; alternatively the method may include inserting the base with the oral retractor (i.e., if the base and oral retractor are pre-assembled or are integrally formed). Any of these methods may include coupling the steerable endotube introducer to the display. Any of these methods may include coupling the display to the stabilizing base.

Any of these methods may include removing the steerable endotube introducer, stabilizing base and oral retractor, and leaving the intubation tube in place. Removing may include using a bougie to remove the steerable endotube introducer, stabilizing base and oral retractor. Deploying the steerable endotube introducer to extend facets may include pushing against tissue to expand access to the patient's trachea using the facets. Inserting the intubation tube may include expanding an anchor on the intubation tube.

For example, a method of inserting an intubation tube may include: attaching an oral retractor and base to a patient's mouth to retract mouth open and establish a stabilizing base; coupling a display to the stabilizing base; inserting a steerable endotube introducer through an opening in the stabilizing base and steer a distal end of the steerable endotube introducer adjacent to the patient's trachea; deploying the steerable endotube introducer to extend facets from a distal end of the steerable endotube introducer; inserting an intubation tube through the steerable endotube introducer and into the patient's trachea; and removing the steerable endotube introducer, stabilizing base and oral retractor, and leaving the intubation tube in place.

All of the methods and apparatuses described herein, in any combination, are herein contemplated and can be used to achieve the benefits as described herein.

### BRIEF DESCRIPTION OF THE DRAWINGS

Novel features of examples described herein are set forth with particularity in the appended claims. A better understanding of the features and advantages of the examples may be obtained by reference to the following detailed description that sets forth illustrative examples and the accompanying drawings.

FIG. 1A schematically illustrates one example of an apparatus (e.g., system) for intubating a patient as described herein.

FIG. 1B schematically illustrates an example of a system for intubating a patient, shown engaged with the patient.

FIG. 2A is a simplified diagram of a system for intubating a patient, including a base and an endotube introducer, and display coupled to a camera on the endotube introducer.

FIG. 2B shows another simplified diagram of an example of a system for intubating a patient.

FIGS. 3A and 3B show an example of a prototype of a jaw retractor including a base having a tube guide entry, shown in a side view and top view, respectively.

FIG. 4 schematically illustrates one example of a distal end region of an endotube introducer, showing the device with the facets cover the distal end deployed.

FIG. 5A is an example of a cross-section through one example of an endotube introducer.

FIG. 5B is another example of a distal end region (tip region) of an endotube introducer showing the distal end closed by the plurality of facets.

FIG. 6 schematically illustrates one example of an oral retractor as described herein.

FIG. 7 schematically illustrates one example of a bougie as described herein.

FIG. 8 is a schematic illustration of one example of a method of performing intubation using an apparatus as described herein.

FIG. 9 shows another example of an apparatus for intubating a patient that is configured to rapidly and safely intubate a patient.

FIG. 10A illustrates an example of an apparatus for intubating a patient.

FIGS. 10B-10C show side and side perspective views, respectively of an example of an endotube introducer for intubating a patient as described herein.

FIG. 10D shows a partial back view of the endotube introducer shown in FIGS. 10A-10C.

FIG. 11 is an exploded view of one example of an endotube introducer for intubating a patient as described herein.

FIGS. 12A-12H illustrate one example of a method for intubating a patient using an apparatus as described herein.

### DETAILED DESCRIPTION

The insertion of an oral endotracheal tube (also referred to herein as an intubation tube) is a vital lifesaving procedure that is conventionally performed in controlled environments by highly skilled clinicians. However, there are some situations, such as rural, point of service (POS) locations, field hospitals, or battlefield locations that can benefit from insertion of an intubation tube, but lack access to the clinicians qualified to perform these procedures.

Described herein are apparatuses (e.g., devices, systems, etc.) for assisting in the insertion of an intubation tube (“intubation”) in a more controlled, safter manner that may avoid problems with insertion. In the U.S., endotracheal intubation is performed about 15 million times in the operating room setting, 650,000 times at the hospital outside of the operating room including 346,000 times in the emergency department interventions. Despite its frequency, endotracheal intubation is a high-risk procedure, with a significant rate of respiratory complications, hemodynamic instability, and cardiac arrest. These complications include failed intubation (approximately 1 in 1-2000 in the elective setting, ≈1 in 300 during rapid sequence induction (RSI) in the obstetric setting, and ≈1 in 50-100 in the emergency department, intensive care unit (ICU), and pre-hospital setting). Any new technology that improves the safety and success of endotracheal intubation can therefore have a tremendous effect on public health and safety. The apparatuses (e.g., systems) described herein may be configured to address these needs.

For example, described herein are trans-oral endoscopic tubes configured as endotube introducers that will guide the operator to the vocal cords. With the help of a flexible and steerable endoscopic tube, this apparatus (e.g., system) does not require a direct view of the vocal cords, which is the current standard of care with the laryngoscope. Furthermore, the distal end (including the tip) of these apparatuses may move in one or more directions, e.g., moving in a plane of the elongate and bent/curved body of the endotube introducer. Alternatively, in some examples the endotube introducer may move in more than this plane (e.g., up, down, left, and right), which will provide the operator significant ease of finding the vocal cords, and aiming the endotracheal tube through the vocal cords, using its camera system and, in some examples, a build-in artificial intelligence agent (e.g., AI algorithm). In some examples the apparatus (e.g., system) may include a mouth opener and stabilizer. In general the endotube introducer and/or stabilizer may be designed to protect all teeth of the patient during the intubation process, while ensuring no damage to the teeth or lumen of the patient. The console (display) may be an LCD or any other appropriate display. The endotube introducer may include a flexible (or bendable, at least at a bend region or bend joint) endoscopic tube with camera, LED light and optionally built-in suction tubes. The endotube introducer may include a built-in maneuvering mechanism to bend and/or steer the device. In some examples the endotube introducer may be configured to allow the user to move the tip in two directions (vertical and horizontal). This mechanism may be manipulated and guided by one or more controls (e.g., knobs, thumb wheels, tactile buttons, switches, e.g.,) and may optionally include one or more microcontrollers, micromotors, etc. The endotube introducer may also include a distal end region that is configured to prevent damage to the tissue, while allowing controlled displacement of tissue (e.g., epiglottis). In some examples the distal end region of the endotube introducer may include one or more petals or facets at the distal end that may extend and/or expand outwards (in some cases from a first position that closes the distal end of the endotube introducer) to a displace tissue or other material allowing more clear visualization and targeting of the trachea. The system may also or alternatively include a thin endoscopic bougie with a camera which connects to the console and LCD. This boogie may be used to remove the flexible endoscopic tube while keeping the endotracheal tube in place.

In some cases these apparatuses (systems, devices, etc.) may include an oral retractor or oral retractor region that couples with or optionally forms a stable base on which imaging and guidance, for guiding an intubation tube may be coupled. Optionally, in some examples the components of the apparatus (e.g., system) may engage with each other to form the stable base and may use the stable base as a reference for guiding and imaging when placing an intubation tube. The apparatus assembly (oral retractor, base, guide tube/endotube introducer, etc.) may be configured specially to be applied to the patient extremely rapidly and without requiring training. For example, the base may be pre-attached to the oral retractor and/or it may be configured to quickly and securely lock onto the oral retractor once applied to the patient. Similarly the imaging (display) may be pre-couped to the base or may be configured to easily engage (snap onto) the base and may couple wirelessly or via a quick-connect cable to the camera(s) of the endotube introducer and/or bougie.

The apparatus (e.g., the endotube introducer) may be configured to be removed, leaving behind the inserted intubation tube, without disrupting or dislodging the intubation tube. In some examples a bougie may be configured to stabilize the intubation tube after it has been inserted, so that the other components of the apparatus may be removed, leaving behind the intubation tube.

In some examples, these apparatuses include one or more (or all) of: an oral retractor, a base coupled or couplable to the oral retractor, a display coupled or couplable to the base; an endotube introducer configured to be inserted through the opening through the base and into the oral cavity, and optionally an intubation tube configured to be inserted through the endotube introducer, and/or a bougie configured to be inserted through the endotube introducer to stabilize the intubation tube while the endotube introducer, display, base and oral retractor are removed.

The base may include an opening through the base into the oral cavity. This opening may include or may be configured to mate with a tube guide entry. The endotube introducer may include one or more cameras in communication with the display, wherein the endotube introducer and has a steerable distal end.

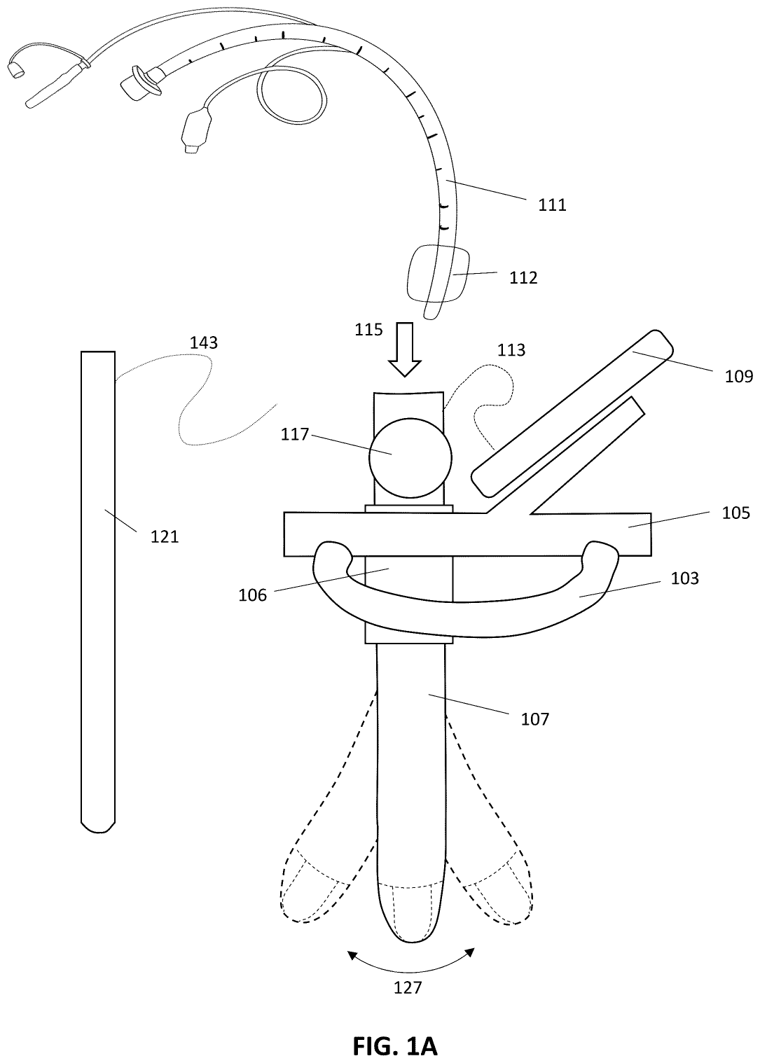

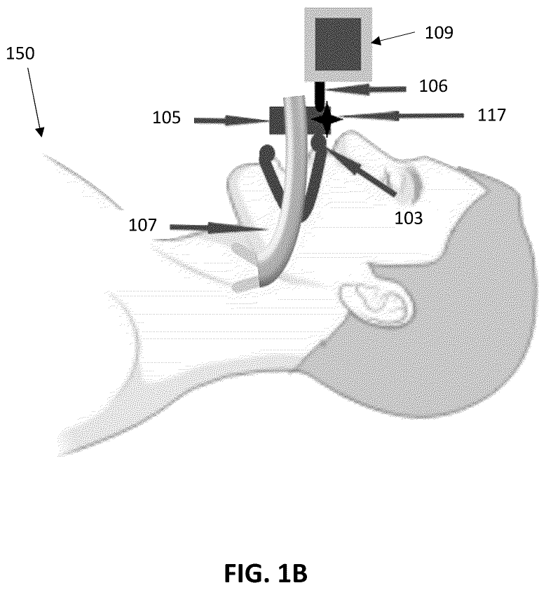

FIG. 1A illustrates one example of an apparatus including at least some of these components. For example, in FIG. 1A the apparatus includes an oral retractor 103 portion (shown schematically from one side). The oral retractor/retractor portion may be, for example, similar to a Jennings Gag, and may be coated or covered in a biocompatible and autoclavable material, such as silicone. The oral retractor may be reusable (e.g., may be sterilized). The oral retractor may include one or more attachment for coupling to a base 105. The base may be coupled magnetically and/or mechanically, e.g., via one or more clips, clamps, etc. The base may extend over and across the patient's mouth when wearing the oral retractor and may include an opening though the base to allow access into the oral cavity. The base may include a central oral opening (e.g., a tube guide entry) that may include a tube 106 or may simply be an opening, into which the endotube introducer and other components may pass through 115 into the patient's mouth.

In FIG. 1A, a display 109 (e.g., LCD screen, touchscreen, etc.) is shown coupled to the base. The display may be adjustable. The display may be coupled to the base prior to attaching the base to the oral retractor, or it may be attached after coupling the base and the oral retractor in variation in which the two are separate components. Note that in some examples the base and oral retractor may be integrated together.

The display may be covered with a protective cover (e.g., a disposable cover, such as a plastic sterile barrier that may be removed and/or replaced and/or sterilized between patients.

FIG. 1A also illustrates one example of an endotube introducer 107 shown inserted into the base 105 through the central oral opening. In this example, the endotube introducer includes a steerable distal end that may move the distal end region in one or more directions 127, by actuating a control, shown as knob 117 on the proximal portion of the endotube introducer projects above the base. In some examples the endotube introducer includes a plurality of tendons that are actuated to pull (or in some examples, push) the distal end region that may be jointed or hinged to allow movement for steering. The distal end region may also include one or more distal-facing (and/or side facing) cameras, light sources, and channels (e.g., irrigation channels, etc.). The endotube introducer may couple to the display via a cable/cord 113 or wirelessly. For example the endotube introducer cameras may be configured to display one or more video images to the display screen wirelessly or via a wired connection. The endotube may include onboard processing (e.g., circuitry for controlling imaging, illumination, and/or steering), and/or may store, transmit and receive data. The endotube introducer may include onboard power, e.g., battery, capacitor, etc. or may be configured to receive power, e.g., through the cabling or connection to the display or to a power source (not shown). In some examples the endotube introducer is unpowered. For example, imaging may be passive (via one or more fiber optics); illumination may be provided by one or more LEDs which may be very low power, and/or may be provided through fiber optic/light pipe coupling to an outside light source (e.g., LED).

The endotube introducer 107 shown in FIG. 1A include a plurality of expandable facets, petals, flaps, etc. that are positioned at the distal end of the endotube introducer and may be driven (via one or more manual or automatic controls on the proximal end of the endotube introducer) to expand outwards (not shown in FIG. 1A). This expansion may help expose the target tissue region, e.g., the trachea, to improve accuracy and precision in placing the intubation tube in the patient. The facets may be, e.g., hinged (in some examples via a living hinge or the like) to open outward. For example, the facets may be configured to expand outward when driven by a wire, rod, etc., or when pulled from a distal region.

In some examples an endotube introducer may have an inner diameter (e.g., ID) that is between about 8 and about 12 mm (e.g., about 10 mm) and an outer diameter that is between about 15 and about 18 mm and a length of between about 7-10 inches (e.g., between about 7-9 inches, about 8 inches, etc.).

Optionally, any of the apparatuses described herein may include or may be configured to operate with an intubation tube 111. Any appropriate intubation tube may be used. In FIG. 1A the intubation tube includes an anchoring balloon 112 at the distal end (and an inflation channel and inlet) and may be sufficiently flexible to be inserted down the endotube introducer once it has been positioned. The intubation tube may be part of a system including all or some of the other components, or it may be separate. For example, the intubation tube may be configured to operate with the apparatuses described herein, including having a custom/specific length and/or a proximal engagement to help guide insertion and/or inflation of the apparatus using the apparatuses described herein. The intubation tube may, for example, include an inner diameter (ID) that is between 6 or 7 mm and may have an OD that is less than the ID of the endotube introducer. The intubation tube may have a length that is long and narrow, e.g., between 20 and 36 inches, e.g., between about 22 and 32 inches, between about 23-37 inches, etc.

Any of these apparatuses may also or additionally include a removal tool, referred to herein as a bougie 121 (or an endoscopic bougie tube, EBT). For example, the bougie may be between 25-48 inches (e.g., between 30-40 inches, between 34-38 inches, etc.) and may have an OD that is just less than the inner diameter of the intubation tube, e.g., by about 1 mm. The bougie may optionally include an imaging (e.g., camera) and illumination (e.g., LED) and may also couple to the display 109 via a wired or wireless connection 143.

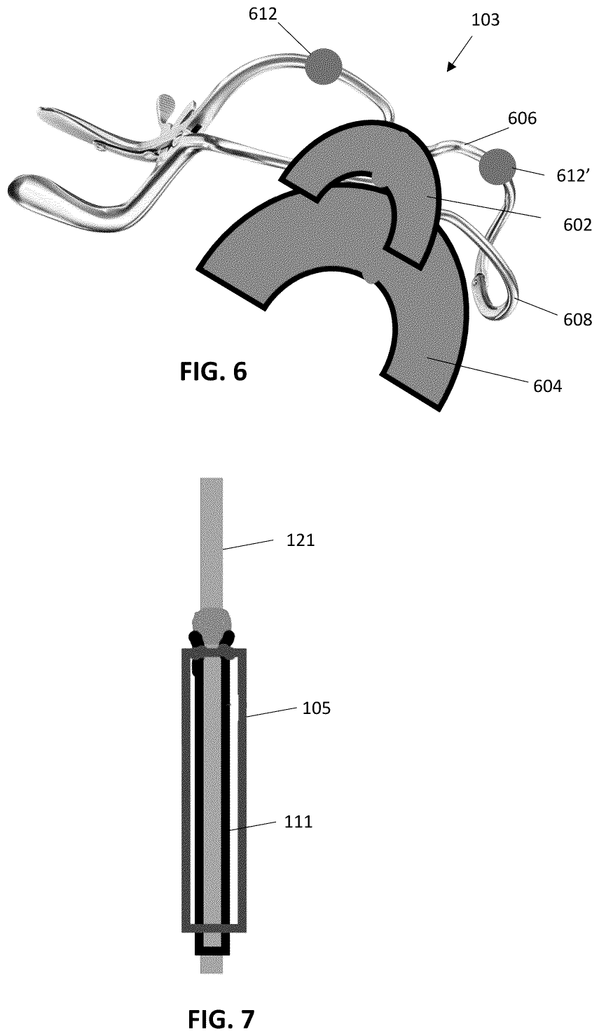

FIG. 1B shows another example of a system for intubating a patient 150. In this example, the system includes a base 105 (e.g., platform), which is connected to an upper arm of an oral retractor 103. The oral retractor includes an upper cavity for holding the upper jaw and/or teeth on a first arm and a lower cavity for holding the lower jaw and/or teeth on the second arm (illustrated in FIG. 6, below). The base also includes or is coupled to an interphase 106 that couples the display 109 (e.g., screen and power unit) to the base. In FIG. 1B, an example of an endotube introducer 107 is also should passing through (and held in position by) the base. The endotube introducer includes a control 117 (e.g., endotube introducer lever arm controller) that may be used to steer the endotube introducer.

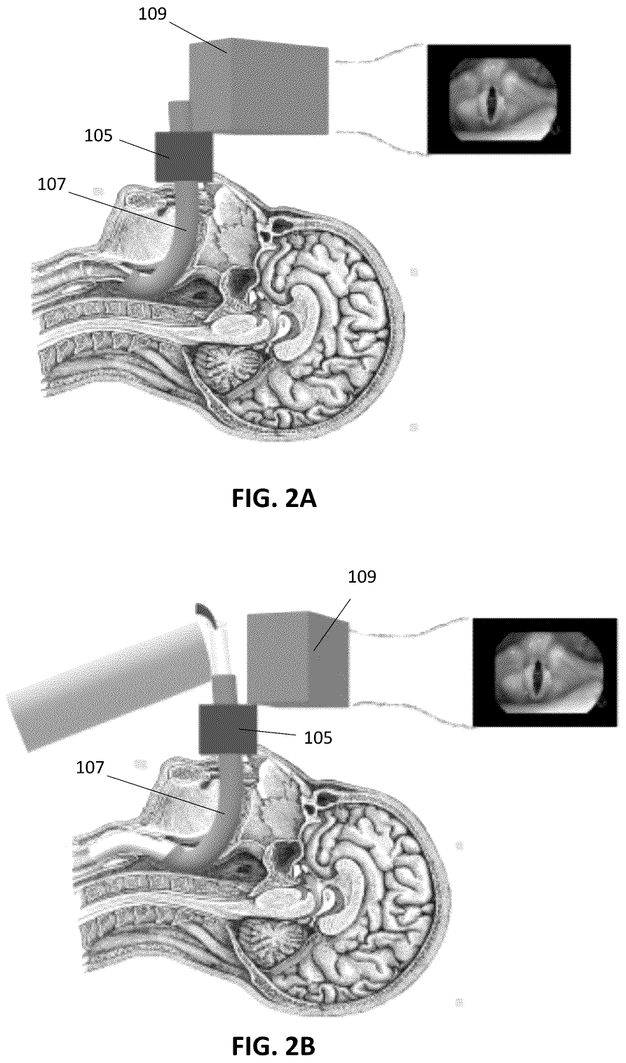

FIGS. 2A-2B illustrate examples of schematics showing possible operation of an apparatus as described herein. In FIG. 2A, an oral retractor mounted to a base 105 includes an opening into the mouth of the patient into which an endotube introducer 107 is inserted. A display 109 shows an image of the vocal cords as the endotube introducer is steered and moved into position, to allow insertion of an intubation tube, as shown in FIG. 2B.

In any of these apparatuses the base may include circuitry (e.g., a processor, memory, etc.) for controlling various components of the apparatus, including imaging and/or steering/guidance of the endotube introducer, etc.

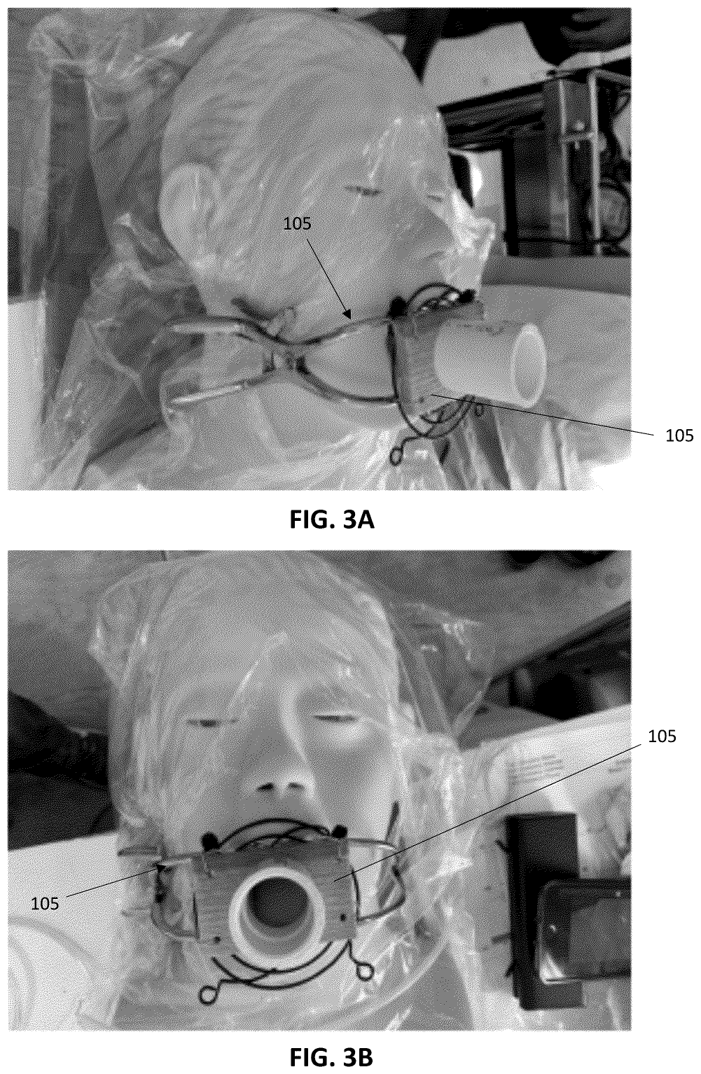

FIGS. 3A and 3B illustrate an example of a model of a patient onto which an oral retractor 105 has been inserted, separating the patient's jaws and holding the teeth apart and away from the central oral opening, as shown. The base 105 in this example includes a tubular projection into the oral cavity and above the base, to which the endotube introducer may be movably and/or securely coupled. The base may also include an attachment for a display (not shown).

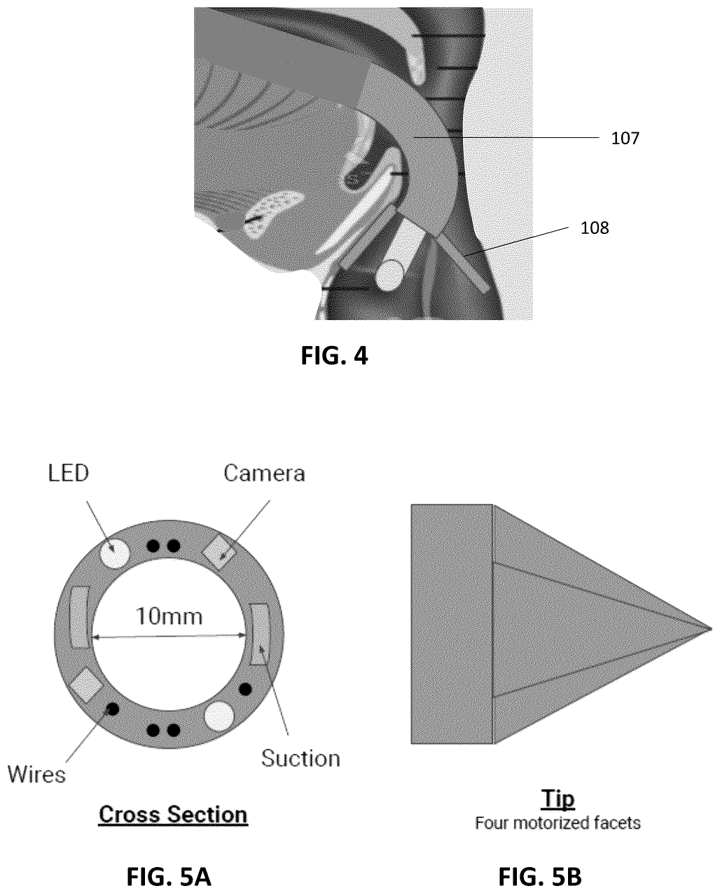

FIG. 4 schematically illustrates an example of an endotube introducer 107 that has been inserted in through the mouth towards the vocal cords, and steered to face the vocal cords, as shown. The endotube introducer also includes multiple facets 108 that are shown open in this schematic example, and may push away the tissue, providing clear access for imaging and/or insertion of one or more additional devices, such as intubation tubes.

FIGS. 5A and 5B illustrate portions of one example of an endotube introducer as described herein. In FIG. 5A a cross-section through a distal end region (or distal end face in some examples) is shown. The example shown in FIG. 5A includes a pair of cameras on opposite sides from each other, as well as a pair of LEDs and a pair of suction ports. The channels for one or more wires are also shown; wires may include steering wires and/or one or more wires for opening/closing or expanding the facets on the distal end of the endotube introducer. In FIG. 5B an example of a distal tip region is shown including four motorized facts that may be opened as described herein. Although they are shown having a pointed/pyramidal structure, the facets may be atraumatic, including curved, rounded, etc. Also the facets are shown closing off the distal end region of the tube, in some examples they do not close entirely but may allow passage of air and/or fluid.

FIG. 6 shows one example of an oral retractor 103 as described herein. In this example the oral retractor includes an upper member 606 to which a first mouthpiece 602 is coupled, and a lower member 608 to which a second mouthpiece 604 is coupled. The oral retractor may include one or more couplers 612, 612′ on the upper or lower (or both) members, to which a base may be coupled. The mouthpieces on the oral retractor may hold the upper and lower jaws open and may also protect the patient's teeth. The mouthpieces are typically sized and shaped to include a wide channel (e.g., a channel having a diameter of 2 cm or greater (3 cm, 4 cm, 5 cm, 6 cm, 7 cm, etc. or more), and are configured to conform to a number of different mouth configurations and sizes. In some examples, the mouthpieces may be adjustable. For example, the upper and/or lower mouthpiece may at least initially slide forward and backwards relative to the upper and lower members, to better accommodate different jaw configurations. In some examples, once the jaw retractor is locked in position, securing the patient's jaws open, the upper and lower jaws may be locked in position, preventing further sliding. In some examples the channel of the mouthpiece may be at least partially open on the lingual and/or occlusal sides of the mouthpiece, so that the buccal (front-facing) surface is protected while allowing different configurations of teeth and/or jaws to fit. In some examples, only the buccal and occlusal sides of the channel are enclosed. The mouthpiece may be formed of a rigid or flexible material, or a combination of the two. For example, the mouthpieces may be formed of a flexible polymeric material, such as a silicone. The mouthpiece may be reinforced; for example a stainless steel reinforcement may be used.

FIG. 7 illustrates one example of a bougie 121, as described herein. In general, the bougie may include an elongate body that is insertable over and/or into an endotracheal tube 111 when the endotracheal tube is supported within an endotube introducer 105, as shown in FIG. 7. As mentioned above, the bougie may be used to assist in removing an endotube introducer while leaving the intubation tube intact and in position after the patient's vitals have stabilized. Optionally, the bougie may include a camera to allow imaging of the position of the intubation tube, e.g., confirming that the intubation tube remains in the correct location while removing the endotube introducer, base and oral spreader. In general, the bougie may be a tubular member that may fit into and support the intubation tube. In FIG. 7, the bougie is inserted into the endotube introducer. The bougie may be solid or may include an airway passage.

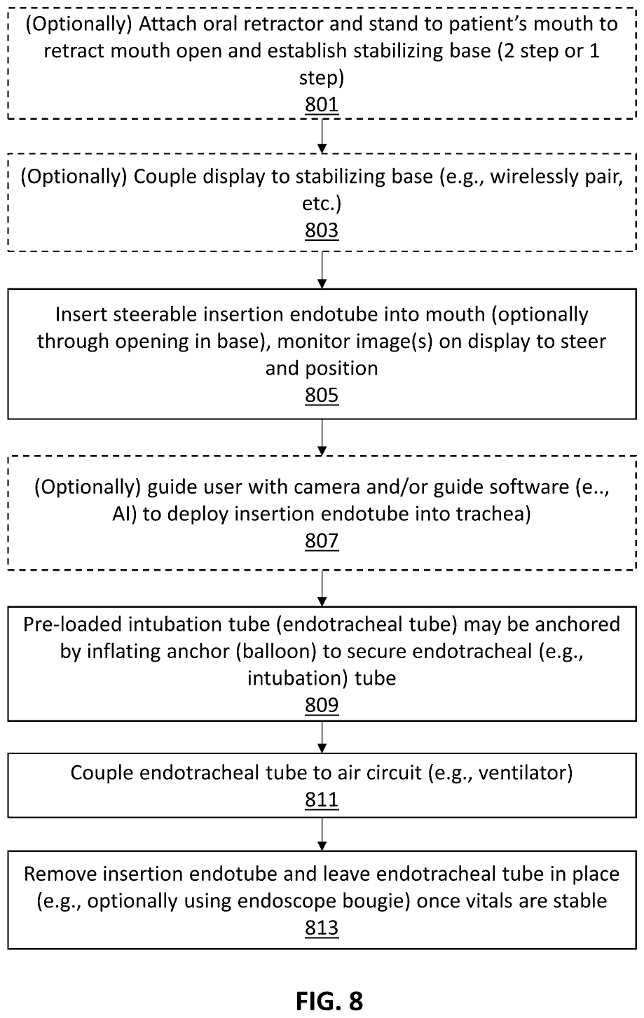

In operation, the apparatuses described herein may be configured to intubate a patient in need thereof. FIG. 8 is chart illustrating one example of a method of intubating a patient, e.g., using an apparatus as described herein. The patient may be positioned on their back. Their head may lay flat or may be tilted back. It is not necessary that the head be tilted back. Optionally, in some example an oral retractor may be attached, holding the patient's teeth and jaws apart, as mentioned above 801. In some examples the oral retractor may be integrated with the endotube introducer; for example, an oral retractor may already be coupled to a base, or the base may be coupled to the oral retractor after it is already attached to the patient. This may form a stabilizing base 801.

The display may (optionally) be coupled to the base 803. In some examples the display may be placed nearby, rather than on the patient supported-base. The display may include a battery or may be coupled to an external power source (e.g., wall source, etc.).

In general, the endotube introducer may be introduced into the patient's mouth 805, in some examples by movably coupled to the base, e.g., by inserting it into the central opening; in some examples the endotube introducer may be directly inserted into the mouth (without requiring a separate base). Optionally, the endotube introducer may be coupled to the display so that video images may be monitored by inserting and positioning the endotube introducer (including steering) within the body. The endotube introducer may be inserted through the patient's mouth until it gets to the pharynx area of the patient's anatomy, and may be positioned near the patient's epiglottis; for example the distal end of the endotube introducer may be maneuvered (e.g., using a “tip tilt” maneuver, e.g., adjusting the bend in the device using the bend control) until the endotube introducer camera is pointing to the patient's trachea. The distal end of the endotube introducer may displace the laryngeal surface of the epiglottis to expose the airway. Once in position, it may optionally be secured onto the base in position 807. The steering component may likewise be locked.

An intubation tube may then be preloaded into the endotube introducer prior to inserting and may be independently advanced once the endotube introducer is in position 809. Alternatively in some examples the endotracheal tube (intubation tube) may be inserted through the endotube introducer until it passes the tip of endotube introducer (e.g., the camera) and enters the into the trachea. Once positioned appropriately, which may be confirmed visually using the display and camera and/or by the guiding agent (the artificial intelligence agent), the intubation tube may be anchored, e.g., by inflating the anchoring balloon to fix it into place. The intubation tube may be pre-coupled to the air circuit 811, or it may be coupled to the air circuit after anchoring the endotracheal tube. The patient is then intubated, and the patient's vitals may be monitored. After the patient's vitals are stable, the apparatus may be removed from around the intubation tube 813. In some examples this may be achieved using the bougie to prevent dislodging the intubation tube when removing the endotube introducer, base, and oral retractor.

The majority of the components included in the apparatuses described herein are sterilizable and reusable. Components may be kept the patient's mouth open and provides a platform some components for and an entry for others.

### EXAMPLES

The endotracheal tube introducer apparatuses (e.g., devices and methods) described herein may enable quick and precise access to hypopharynx to conduct one or more medical procedures when operated by a medical professional. As discussed above, these apparatuses may be easy to use, and relatively inexpensive, allowing use during highly time-sensitive, emergency conditions. Further, these apparatuses may prevent trauma to the teeth and mouth, may be used with minimal training, and may prevent or reduce pressure on the cervical spine during emergency treatment, particularly as compared to existing tools. In general, these methods and apparatuses may avoid the necessity for tracheostomy. Preliminary work has shown that these apparatuses may provide quick access to a patient's hypopharynx to intubate the patient with no adverse outcome in a manner that is five or more times faster than currently available devices.

In general, these apparatuses may include an introducer that is pre-loaded with an endotracheal tube and is equipped with a high-resolution vision system that provides guidance (e.g., via a trained machine learning agent) and in-situ tip adjustment which reduces or eliminates the need to clear the path through the oropharyngeal region. These apparatuses may be used in any appropriate medical or emergency setting, including, but not limited to emergency medical services, hospitals, operating rooms, outpatient clinics, war fields, and any airway emergency medical procedures in unequipped settings. The endotube introducers described herein may allow quick and precise access to hypopharynx and may allow a medical professional to conduct various operations.

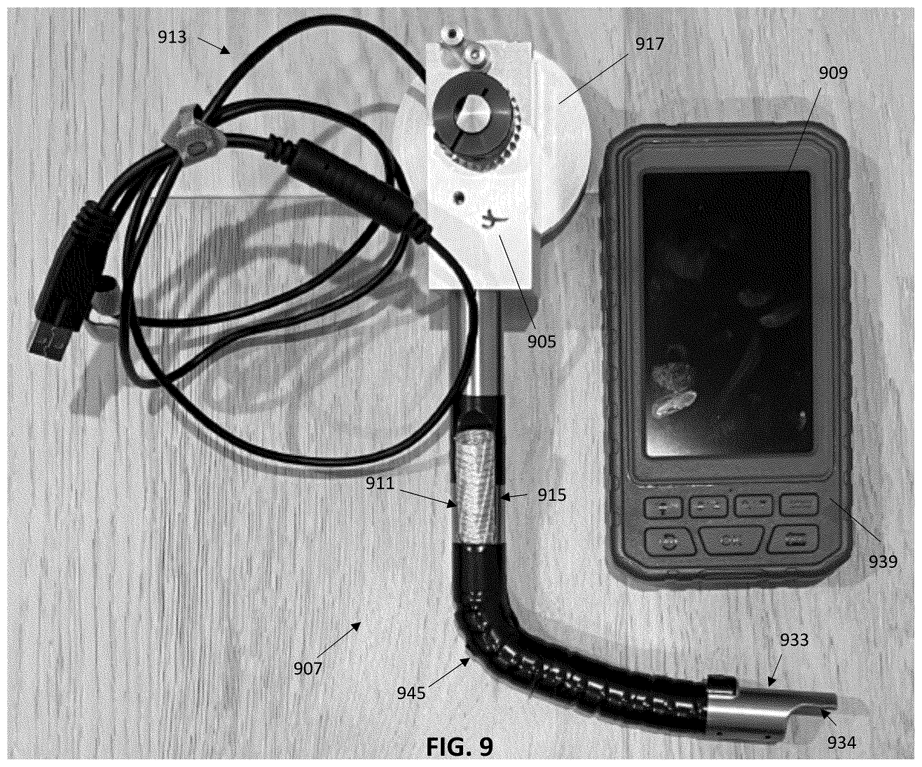

FIG. 9 illustrates one example of an apparatus, configured as a system that includes an endotube introducer 907, a display 909, a connecting cable 913 and may include or be preloaded with an endotracheal tube 911 (visible in the cut-away region 915 through the elongate body of the endotube introducer). The example shown in FIG. 9 includes a pre-loaded endotracheal tube within the endotube introducer, and a high-resolution vision system. The endotube introducer is configured to allow an in-situ adjustment of the distal end region (e.g., tip) which reduces the need to clear the path through the oropharyngeal region. In general, these apparatuses, including the endotube introducer may formed of materials that are biocompatible and suitable for tissue contact, e.g., within the oral and respiratory cavity. These apparatuses may be used as part of an intubation procedure that has traditionally required establishing a direct line-of-sight to the patient's trachea using a laryngoscope.

In FIG. 9, the endotube introducer includes a distal end 933 that is configured to extend distally from an elongate body of the endotube introducer. The distal end includes an extension or tongue region 934 that may be soft, but provide sufficient support to displace (e.g., elevate) the epiglottis when the tip steering control 917 (e.g., bend control) is activated. In FIG. 9 the bend control 917 is configured to increase the bend at the bend region 945 approximately halfway up the length of the elongate body of the endotube introducer. The endotube introducer also includes a proximal base region 905 that may be configured to insert only partially into the mouth or oral cavity and in some cases may engage with a separate base and/or retractor coupled to the patient's mouth. This separate base may be optional.

In FIG. 9 the cord 913 may couple directly to the display 909 and one or more processors 939. Alternatively in some examples the camera(s), e.g., at the distal end region of the endotube introducer, may wireless connect to one or more processors 939. The one or more processors may process image(s) from one or more cameras that are on (or coupled with) the distal end 933 and may include video image processing and/or storage. Optionally, in some cases the one or more processors, may be configured to locally or remotely process the images and provide guidance to the user as to how to steer and/or position the distal end region of the endotube introducer so that the endotracheal tube may be positioned and anchored within the trachea. Any of these apparatuses and methods may include signal processing of the video signal(s) so that the images displayed with the correct contrast, brightness, etc. In any of these methods and apparatuses the images may be configured to be cropped, scaled, filtered, etc. In some example the one or more processors may be configured to mark and/or label the images, e.g., to indicate region(s) such as the epiglottis, airway, etc.

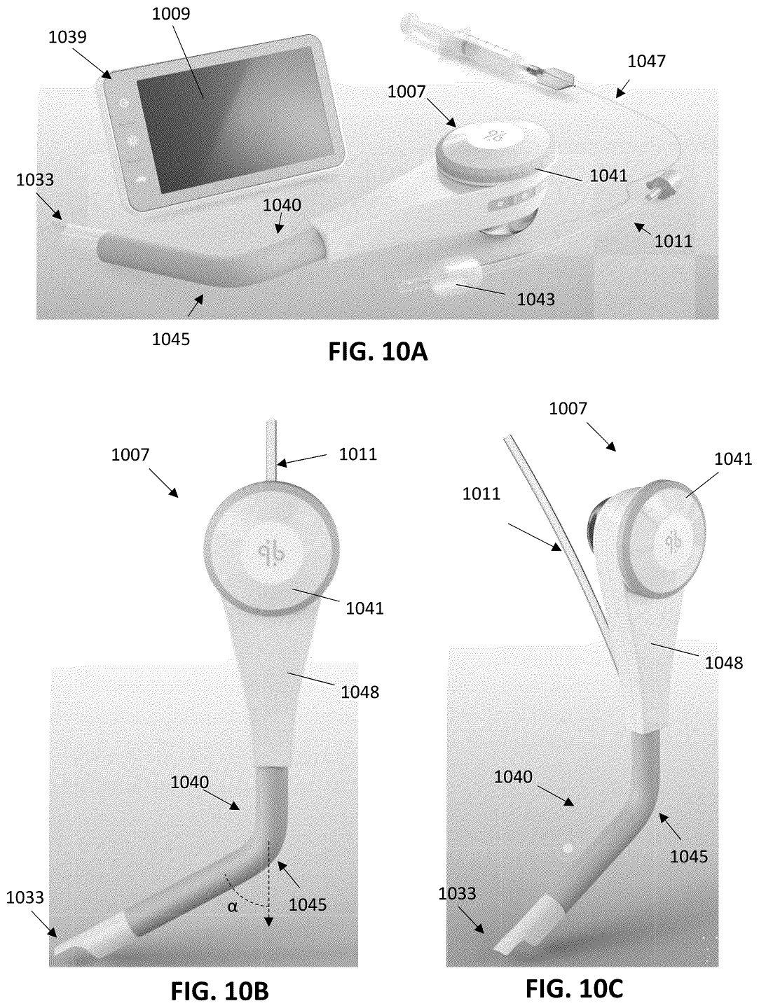

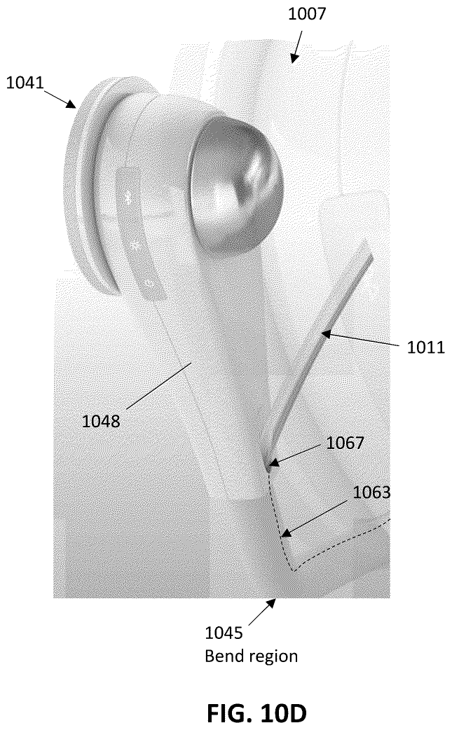

FIGS. 10A-10D illustrate another example of an apparatus, configured as a system, for intubating a patient as described herein. In FIG. 10A the system includes an endotube introducer 1007 that is configured to be inserted through an oral cavity (e.g., mouth, esophagus, etc.) and into a tracheal region of a body. The endotube introducer may generally include an elongate body 1040 having a lumen extending at least partially along the length (particularly at the distal end region), and a bend region 1045 on the elongate body. The endotube introducer may also include (or may be configured to couple with) a camera (not visible in FIGS. 10A-10C) at a distal end region 1033. The endotube introducer also includes a bend control 1041 at a proximal end region configured to alter the bend of the bend region.

In FIG. 10A, the system also includes a display 1009 that is configured to receive and display output from the camera. The system may further include one or more processors 1039. Although the examples shown in FIGS. 9 and 10A include the processor(s) integral with the display 1009, the one or more processors may be part of the endotube introducer and/or separate from the display and/or endotube introducer. The one or more processors and/or display may be wireless connected to the camera and/or endotube introducer.

In FIG. 10A the display 1009 and processor 1039 are shown as an included part of the apparatus. In some examples the display and/or processor may be part of a device such as a smartphone, pad, laptop, etc. that may be paired with the endotube introducer and other components. Thus, the user may supply this component and may install software for use with the endotube introducer to perform the functions (e.g., display, guidance, etc.) as described herein.

FIG. 10A also shown one example of an endotracheal tube, e.g., intubation tube, 1011, that may be used with the endotube introducer, including may be preloaded into the endotube introducer. The endotracheal tube 1011 includes an anchor 1043, configured as an inflatable balloon in FIG. 10A. The anchor may be actuated and expanded by applying fluid (e.g., saline, air, etc.) via an inflation line 1047. The endotracheal tube is generally configured to be coupled to an air circuit (e.g., respirator, etc.) and in some cases may be pre-coupled to the air circuit. Thus, the intubation tube 1011 may be preloaded within the lumen of the insertion endoscope, and the intubation tube may be configured to be advanced distally out of the endotube introducer.

FIGS. 10A-10D illustrate an example of an endotube introducer 1007 from different views. The endotube introducer include a proximally-located bend control 1041 and an elongate body 1040 extending from the proximal base region 1048 that may be gripped and/or may be configured to prevent damage to the mouth/teeth of the patient. The elongate body in this example is pre-bent, to have a resting bend angle from a bend region 1045 that is approximately mid-way along the length of the elongate body 1040. For example, the endotube introducer may bend starting at about between about 7 cm (e.g. 8 cm, 9 cm, 10 cm, 11 cm, 12 cm, 13 cm, 14 cm, 15 cm, etc.) and about 25 cm (e.g. 24 cm, 23 cm, 22 cm, 21 cm, 20 cm, 19 cm, etc.) of the distal end region of endotube introducer 1033. The resting bend angle (α) may be, e.g., between about 25 degrees and about 50 degrees. In the example shown in FIG. 10B the resting bend angle (α) is about 40 degrees relative to a longitudinal axis of the elongate body in the more proximal region. This bend angle may be increased/decreased by the control 1041 (bend control).

The distal end region 1033 in this example may be formed of a different material than the elongate length, and may be configured to prevent damage to the airway. For example the distal end region may be rounded and/or formed of a softer material (e.g., a material having a lower durometer) than the elongate body 1040 of the endotube introducer. The distal end region may house or include one or more cameras that are distal-facing and configured to view the lumen in which the endotube introducer is inserted.

The endotube introducer 1007 shown in this example is pre-loaded with an endotracheal tube 1011 at least partially housed within the lumen of the endotube introducer. As shown in FIGS. 10C and 10D, the endotracheal tube may be inserted into an insertion region 1067 that may be part of the base 1048 of the endotube introducer. The insertion region 1067 may be funnel-shaped to guide insertion of the endotracheal tube into the distal end region of the endotube introducer. In FIG. 10D the elongate body of the endotube introducer includes a lateral side opening region 1063 extending from the insertion region 1067 to the distal end of the endotube introducer. This longitudinal side opening may provide access into the lumen of the endotube introducer and may be used to remove the endotracheal tube after it is anchored and positioned. In some cases the endotracheal tube may be pulled out of this longitudinal side opening of the endotube introducer after being anchored in place. The longitudinal side opening 1063 may be closed but can be formed of a material (e.g. polymer) that can be displaced by pulling the endotracheal tube. The longitudinal side opening 1063 may be a slit or cut. The longitudinal side opening may be closed or held closed until removal of the endotracheal tube is desired.

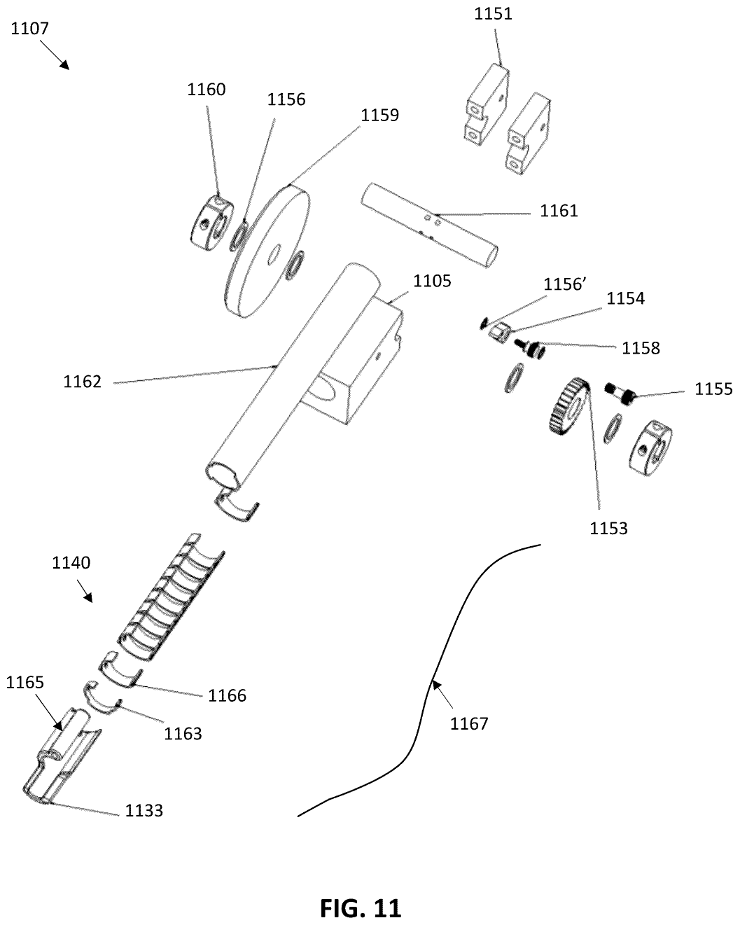

FIG. 11 shows an example of an exploded view of an endotube introducer such as the one shown in FIG. 9. In FIG. 11 the endotube introducer 1107 includes a distal end region 1133 that couples with a camera 1165, and is attached at the distal end of the elongate body 1140. At least a portion of the elongate body, such as the bend region, may be formed of segments 1165 (e.g. vertebra) that may allow bending. A proximal control (e.g., dial, knob, etc.) 1159 may be connected to the cable 1167 or tendon that passes through the segments and connects to a more distal attachment 1163. The cable 1167 may coupled to a steering cable roll-up rod 1161 in this example, which may be held to a base 1105 (e.g., adjustment block) by one or more rod holders 1151 that allow the rod to roll as the control (knob) 1159 is rotated. The base 1105, control 1159 and holders 1151 may be coupled together at the proximal end of the device by a plurality of washers 1156, 1156′, collars 1160 and screws 1155, 1158. The control may also be connected to one or more gears 1153 and pawls 1154 that may hold the position of the bend, even against a relatively large force (e.g., 2 N, 3 N, 4 N, 5 N, 6 N, 7 N, 8 N, 9 N, 10 N, etc.) resisting bending of the elongate body. The elongate body may be at least partially covered by a coverage 1162.

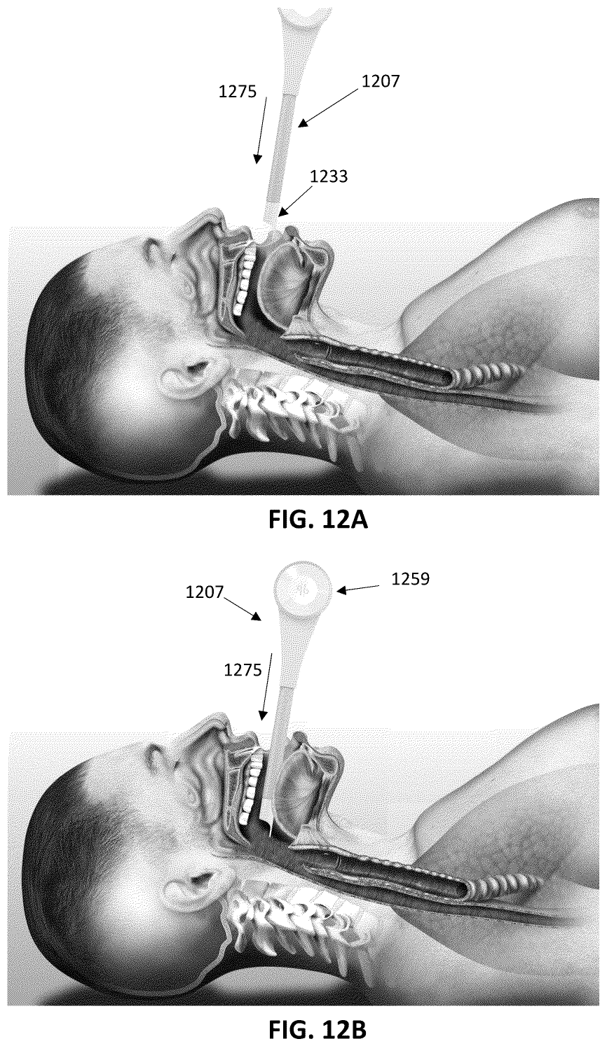

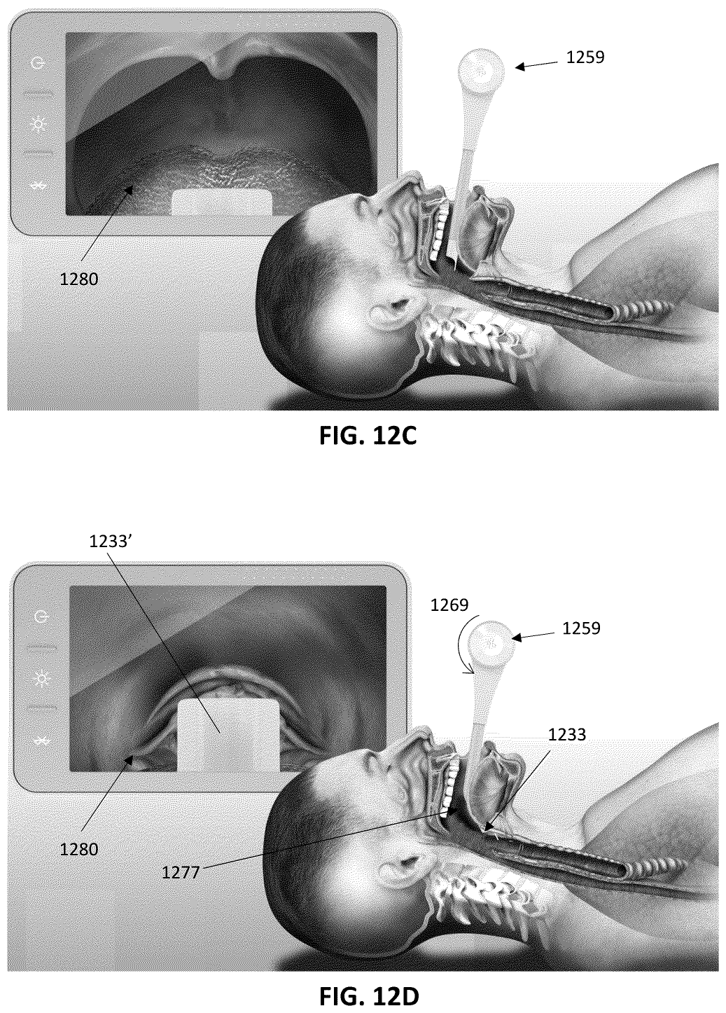

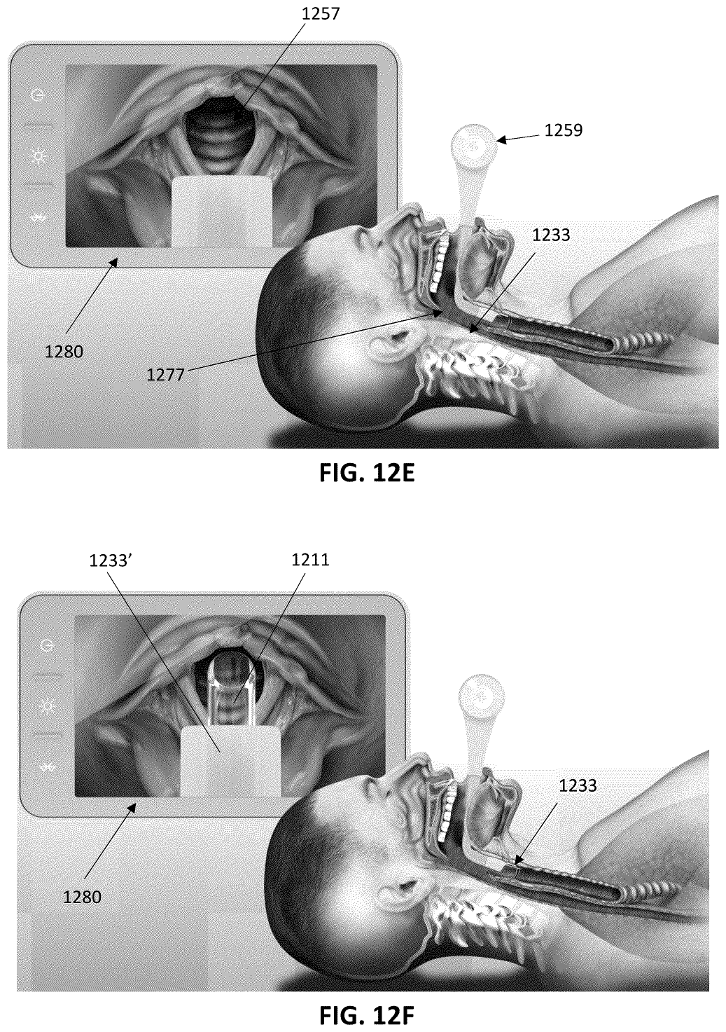

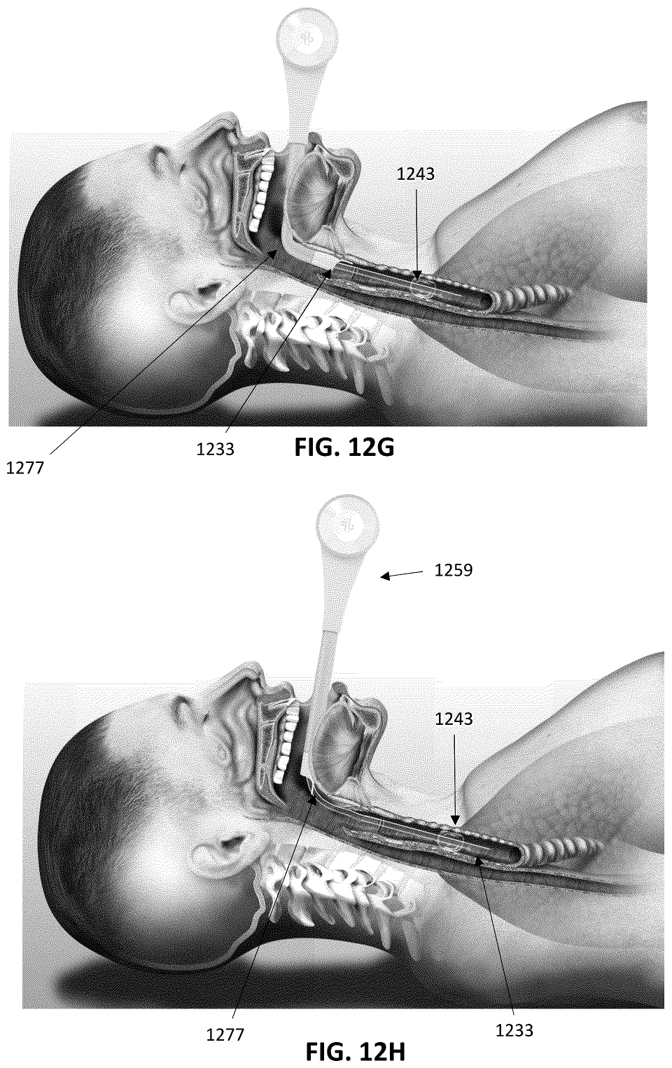

FIGS. 12A-12H illustrate one example of a method of using an apparatus as described herein to quickly and safely introduce an endotracheal tube into a patient's trachea. In FIG. 12A the patient is shown lying supine and the endotube introducer 1207 is inserted 1275 into the subject mouth, between the teeth, distal end 1233 first, over the patient's tongue. In some examples the elongate body of the device may be inserted straight as shown; alternatively in some example the elongate body may be pre-bent as described above. Alternatively the bend may be controlled during insertion by the bend control (e.g., knob) 1259 at a proximal end of the device. The endotube introducer 1207 may be placed over the patient's tongue towards the oropharynx and hypopharynx with the preloaded endotracheal tube already held within the lumen of the endotube introducer as shown in FIG. 12B.

During insertion the camera may be used to visualize (to a display 1280) a shown in FIG. 12C what is ahead of (distal to) the distal end of the endotube introducer. During the procedure the endotube introducer may be moved forwards so that the tip, viewed on the camera, is positioned beneath the laryngeal surface of the epiglottis, as shown in FIG. 12D. The control 1259 may be activated 1269 (e.g., by rotating in this example) to bend the distal region 1233 of the endotube introducer from a bend region 1277 towards the airway. In FIG. 12D the distal end region 1233 is moved by the control so that it elevates the epiglottis. For example the endotube introducer may be advanced until the tip 1233, 1233′ of the endotube introducer, which is visible on the display, is positioned beneath the laryngeal surface of the epiglottis. The tip 1233 may then be rotated to elevate the epiglottis and expose the airway 1257, as shown in FIG. 12E. As the vocal cords are viewed, e.g., on the display 1280, the preloaded endotracheal tube 1211 may be advanced distally within the endotube inserter so the endotracheal tube is pushed through the vocal cords into the trachea. The endotube inserter may be held in position during this process. After the endotracheal tube placement is confirmed (e.g., visually using the display in some examples), the anchor 1243 on the endotracheal tube may be expanded, e.g., in some examples, inflated. This is shown in FIG. 12G. Once the endotracheal tube is anchored within the trachea, the endotube inserter may be withdrawn, as shown in FIG. 12H, and removed from over the endotracheal tube. The endotracheal tube may be connected to an air circuit to apply the patient with air through the endotracheal tube.

When a feature or element is herein referred to as being on another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and examples such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. A system comprising:
an endotube introducer configured to be inserted through an oral cavity into a tracheal region of a body, the endotube introducer comprising an elongate body having a lumen extending therethrough and a bend region on the elongate body, a camera at a distal end region, and a bend control at a proximal end region configured to alter a bend of the bend region;
a display configured to receive and display output from the camera;
an intubation tube preloaded within the lumen of the endotube introducer, the intubation tube having an expandable anchor at a proximal end region, wherein the intubation tube is configured to be advanced distally out of the endotube introducer.

2. The system of claim 1, wherein the bend control is configured to bend the bend region to 70 degrees or more relative to a longitudinal axis of the elongate body.

3. The system of claim 1, wherein the bend region has a minimum set bend of between about 25 and 50 degrees.

4. The system of claim 1, wherein the bend control is configured to bend the bend region against 2 Newtons (N) or greater of force.

5. The system of claim 1, wherein the bend region is between 7 and 20 cm from the distal end region of the endotube introducer.

6. The system of claim 1, wherein the distal end region of the endotube introducer comprises one or more projections configured to atraumatically displace tissue.

7. The system of claim 1, wherein the endotube introducer is wirelessly coupled to the display.

8. The system of claim 1, further comprising a longitudinal side opening through the body and into the lumen.

9. The system of claim 1, wherein the distal end region is constrained to bend in a first plane that is parallel with the elongate body.

10. The system of claim 1, further comprising one or more processors, wherein the one or ore more processors comprises a non-transitory computer-readable medium including instructions that, when executed by the one or more processors, cause the one or more processors to perform a method comprising: presenting guidance information on the display to guide a user to insert the endotube introducer.

11. The system of claim 1, wherein the bend control comprises a dial.

12. A system comprising:
an endotube introducer configured to be inserted through an oral cavity into a tracheal region of a body, the endotube introducer comprising an elongate body having a lumen extending therethrough and a bend region between 7 cm and 20 cm from a distal end of the elongate body that is configured to increase a bend of the elongate body to 70 degrees or more relative to a longitudinal axis of the elongate body, a camera at a distal end region, and a bend control at a proximal end region configured to bend or straighten the elongate body at the bend region; and
a display configured to receive and display output from the camera;
wherein the lumen is configured to slidably hold an intubation tube preloaded within the lumen of the endotube introducer so that the intubation tube may be advanced distally out of the endotube introducer.

13. The system of claim 12, wherein the bend region is pre-bent to an initial angle of between about 25 and 50 degrees relative to the longitudinal axis of the elongate body.

14. The system of claim 12, wherein the bend region has a minimum set bend of between about 25 and 50 degrees.

15. The system of claim 12, wherein the bend control is configured to bend the bend region against 2 Newtons (N) or greater of force.

16. The system of claim 12, wherein the bend region is between 7 and 20 cm from the distal end of the endotube introducer.

17. The system of claim 12, wherein the distal end region of the endotube introducer comprises one or more projections configured to atraumatically displace tissue.

18. The system of claim 12, wherein the endotube introducer is wirelessly coupled to the display.

19. The system of claim 12, further comprising a longitudinal side opening through the body and into the lumen.

20. The system of claim 12, wherein the distal end region is constrained to bend in a first plane that is parallel with the elongate body.

21. The system of claim 12, further comprising one or more processors, wherein the one or ore more processors comprises a non-transitory computer-readable medium including instructions that, when executed by the one or more processors, cause the one or more processors to perform a method comprising: presenting guidance information on the display to guide a user to insert the endotube introducer.

22. The system of claim 12, wherein the bend control comprises a dial.

23. A method of inserting an intubation tube, the method comprising:
inserting a distal end region of an endotube introducer having an elongate body through to a patient's mouth, wherein the endotube introducer is preloaded with an endotracheal tube;
advancing the distal end region of the endotube introducer into the patient, while imaging a region distal to the distal end region from a camera on the distal end region, to position the distal end region beneath a laryngeal surface of an epiglottis;
activating a control on the endotube introducer to increase a bend of the elongate body so that the distal end region elevates the epiglottis;
advancing the endotracheal tube distally out of the endotube introducer and into the patient's trachea;
anchoring the endotracheal tube within the trachea; and
removing the endotube introducer from the patient leaving the endotracheal tube within the trachea.

24. The method of claim 23, wherein anchoring comprises expanding an expandable balloon.

25. The method of claim 23, further comprising connecting the endotracheal tube to an air circuit.

26. The method of claim 23, wherein activating the control comprises increasing the bend of the elongate body from an angle of between 25 and 50 degrees relative to a longitudinal axis of the elongate body to an angle of greater than 70 degrees relative to the longitudinal axis of the elongate body.

27. The method of claim 23, wherein activating the control comprises increasing the bend of the elongate body at a bend region that is between 7 cm and 20 cm from a distal end of the elongate body.

28. The method of claim 23, further comprising assisting, using a guidance sub-system associated with the endotube introducer, in controlling the positioning of the distal end region.

29. A method of inserting an intubation tube, the method comprising:
inserting a distal end region of an endotube introducer having an elongate body through to a patient's mouth, wherein the endotube introducer is preloaded with an endotracheal tube;
advancing the distal end region of the endotube introducer into the patient, while imaging a region distal to the distal end region from a camera on the distal end region, to position the distal end region beneath a laryngeal surface of an epiglottis, wherein a control on the endotube introducer is controlled to increase a bend of the elongate body so that the distal end region elevates the epiglottis;
advancing the endotracheal tube distally out of the endotube introducer and into the patient's trachea;
anchoring the endotracheal tube within the trachea; and
removing the endotube introducer from the patient leaving the endotracheal tube within the trachea.
